(19) United States
(12) Patent Application Publication
(10) Pub. No.: US 2017/0115265 A1
(43) Pub. Date: Apr. 27, 2017

(54) Use of Visibly Detectable Compounds as Performance Reference Compounds in Passive Sampling Devices

(71) Applicant: Geosyntec Consultants, Inc., Boca Raton, FL (US)

(72) Inventor: Jason Michael Conder, Laguna Niguel, CA (US)

(21) Appl. No.: 15/299,580

(22) Filed: Oct. 21, 2016

Related U.S. Application Data
(63) Continuation of application No. PCT/US16/57863, filed on Oct. 20, 2016.
(60) Provisional application No. 62/245,346, filed on Oct. 23, 2015.

(51) Int. Cl. G01N 33/18

(52) U.S. Cl. CPC G01N 33/1826

(57) ABSTRACT

In some embodiments, the invention provides a visibly detectable performance reference compound for use in a passive sampling device to detect an analyte of interest (and/or the amount of the analyte of interest) in an environment. In some embodiments, the invention provides a passive sampling device comprising a visibly detectable performance reference compound for use in detecting an analyte of interest (and/or the amount of the analyte of interest) in an environment. In some embodiments, the invention provides a kit comprising a first visibly detectable performance reference compound and a second visibly detectable performance reference compound for use in a passive sampling device to detect an analyte of interest (and/or the amount of the analyte of interest) in an environment. In some embodiments, invention provides a method to detect an analyte (and/or the amount of the analyte) in an environment using a passive sampling device comprising a visibly detectable performance reference compound.

### CROSS-REFERENCE TO RELATED APPLICATION

This application claims benefit from International application serial no. PCT/US16/57863 filed Oct. 20, 2016 and from U.S. provisional application Ser. No. 62/245,346 filed Oct. 23, 2015, the entire contents of each of which is hereby incorporated by reference.

### TECHNICAL FIELD

The present invention relates to the field of analyzing surface water, aquatic sediment, and soil.

### BACKGROUND ART

Over the past decade, academic and commercial research have demonstrated that measurements made using Passive Sampling Devices (PSDs) represent the most precise and accurate estimates of available organic compounds in aquatic sediment, surface water, and soil (USEPA, “Sediment Assessment and Monitoring Sheet #3: Guidelines for using passive samplers to monitor organic contaminants at Superfund sediment sites”. OSWER Directive 9200.1-110FS, 2012; Cui et al., Environ. Pollut. 172:223-234, 2013; Mayer et al., Integr. Environ. Assess. Manag. 10:197-209, 2014). PSDs are solid devices that are left in contact with aquatic sediment, water, or soil for a period of time, whereupon they absorb organic molecules or metals. After exposure, PSDs are then retrieved and analyzed.

Non-limiting examples of PSDs for sampling organic compounds include devices comprised of polyethylene or polyoxymethylene, or devices incorporating polydimethylsiloxane, such as Solid Phase Microextraction (SPME) fibers (OSWER Directive 9200, 1-110FS, U.S. Environmental Protection Agency, December 2012). PSDs, in general, absorb only compounds that are mobile and bioavailable in the environment, and are considered to be much more accurate measures of chemical availability and potential risk as compared to traditional chemical methods (see USEPA, 2012, supra; Cui et al., supra; Mayer et al., supra). The improvement in accuracy provided by the use of PSDs is considered to offer a more accurate estimation of the risks of chemical contamination, potentially avoiding conservative and expensive remediation or treatment.

However, it would be useful to further optimize PSDs to improve their effectiveness and their ease of use.

### SUMMARY OF THE EMBODIMENTS

In some embodiments, the invention provides a visibly detectable performance reference compound that be used in a PSD to optimize the ease of use and effectiveness of the PSD.

Accordingly, in a first aspect, the invention provides a visibly detectable performance reference compound for use in a passive sampling device to detect an analyte of interest (and/or the amount of the analyte of interest) in an environment.

In another aspect, the invention provides a passive sampling device comprising a visibly detectable performance reference compound for use in a passive sampling device to detect an analyte of interest (and/or the amount of the analyte of interest) in an environment.

In some embodiments, the analyte of interest is an organic molecule.

In some embodiments, the environment is selected from the group consisting of liquid, solid, and gas. In some embodiments, the environment is a soil or a sediment. In some embodiments, the environment is a biological fluid including, for example, urine, blood, and other fluids (e.g., interstitial fluids). In some embodiments, the biological fluid can be taken out of an organism (e.g., a blood sample taken from a human organism). In some embodiments, the biological fluid is within an organism.

In some embodiments, the compound has a first octanol/water partition coefficient (K_{OW}) or first log K_{OW} and the analyte has a second K_{OW} or second log K_{OW}, wherein the first K_{OW} and the second K_{OW} are between about 0.01% to about 10% of each other, or wherein the first log K_{OW} and the second log K_{OW} are between about 0.01% to about 10% of each other. In some embodiments, the first K_{OW} and the second K_{OW} are between about 0.01% to about 5% of each other, or the first log K_{OW} and the second log K_{OW are between about} 0.01% to about 5% of each other. In some embodiments, the first K_{OW} and the second K_{OW} are between about 0.01% to about 1% of each other or the first log K_{OW} and the second log K_{OW} are between about 0.01% to about 1% of each other.

In some embodiments, the compound has a first PSD/water partition coefficient and the analyte has a second PSD/water partition coefficient, wherein the first PSD/water partition coefficient and the second PSD/water partition coefficient are between about 0.01% to about 10% of each other, or wherein the first PSD/water partition coefficient and the second PSD/water partition coefficient are between about 0.01% to about 5% of each other, or wherein the first PSD/water partition coefficient and the second PSD/water partition coefficient are between about 0.01% to about 1% of each other.

In some embodiments, the visibly detectable performance reference compound is not radioactive. In some embodiments, the visibly detectable performance reference compound is not toxic.

In some embodiments, the visibly detectable performance reference compound is absorbable by a material selected from the group consisting of acetate, polyacrylate, silicone rubber, low-density polyethylene (LDPE), polyethylene (PE), polyoxymethylene (POM), polydimethylsiloxane (PDMS), polyethersulfone (PES), or a combination of one or more of the foregoing, and/or solid phase microextraction fibers coated with such sorbents.

In some embodiments, the visibly detectable performance reference compound is hydrophobic.

In some embodiments, the visibly detectable performance reference compound is hydrophilic.

In some embodiments, the visibly detectable performance reference compound emits a wavelength from about 0.01 nanometers to about 1.0 micrometers.

In some embodiments, the visibly detectable performance reference compound is visibly detectable by an ultraviolet/visible light detector.

In some embodiments, the visibly detectable performance reference compound emits a wavelength from about 10 nanometers to about 400 nanometers.

In some embodiments, the compound emits a wavelength from about 250 nanometers to about 450 nanometers.

In some embodiments, the visibly detectable performance reference compound is visibly detectable by an infrared light detector.

In some embodiments, the visibly detectable performance reference compound emits a wavelength from about 700 nanometers to about 1 micrometer.

In some embodiments, the visibly detectable performance reference compound is visible to a human eye.

In some embodiments, the visibly detectable performance reference compound emits a wavelength from about 300 nanometers to about 700 nanometers (e.g., from about 390 nm to about 700 nm).

In another aspect, the invention provides a kit comprising a first visibly detectable performance reference compound and a second visibly detectable performance reference compound for use in a passive sampling device to detect an analyte of interest (and/or the amount of the analyte of interest) in an environment, the first compound having a first octanol/water partition coefficient (K_{OW}), the second compound having a second K_{OW}, and the analyte having a third K_{OW}, wherein the third K_{OW} is between the first K_{OW} and the second K_{OW}.

In another aspect, the invention provides a kit comprising a first visibly detectable performance reference compound and a second visibly detectable performance reference compound for use in a passive sampling device to detect an analyte of interest (and/or the amount of the analyte of interest) in an environment, the first compound having a first log octanol/water partition coefficient (log K_{OW}), the second compound having a second log K_{OW}, and the analyte having a third log K_{OW}, wherein the third log K_{OW} is between the first log K_{OW} and the second log K_{OW}.

In another aspect, the invention provides a kit comprising a first visibly detectable performance reference compound and a second visibly detectable performance reference compound for use in a passive sampling device to detect an analyte of interest (and/or the amount of the analyte of interest) in an environment, the first compound having a first PSD/water partition coefficient, the second compound having a second PSD/water partition coefficient, and the analyte having a third PSD/water partition coefficient, wherein the third PSD/water partition coefficient is between the first PSD/water partition coefficient and the second PSD/water partition coefficient.

In some embodiments, the compound (or at least one of the first visibly detectable performance reference compound and the second visibly detectable performance reference compound of the kits described herein) is absorbable by a material selected from the group consisting of acetate, polyacrylate, silicone rubber, low-density polyethylene (LDPE), polyethylene (PE), polyoxymethylene (POM), polydimethylsiloxane (PDMS), polyethersulfone (PES), or a combination of one or more of the foregoing, and/or solid phase microextraction fibers coated with such sorbents. In some embodiments, the compound (or at least one of the first visibly detectable performance reference compound and the second visibly detectable performance reference compound of the kits described herein) is absorbable by a material having an adsorption property and/or desorption property similar to a material selected from the group consisting of acetate, polyacrylate, silicone rubber, low-density polyethylene (LDPE), polyethylene (PE), polyoxymethylene (POM), polydimethylsiloxane (PDMS), polyethersulfone (PES), or a combination of one or more of the foregoing and/or solid phase microextraction fibers coated with such sorbents.

In some embodiments, the compound (or at least one of the first visibly detectable performance reference compound and the second visibly detectable performance reference compound of the kits described herein) is hydrophobic. In some embodiments, the compound (or at least one of the first visibly detectable performance reference compound and the second visibly detectable performance reference compound of the kits described herein) is hydrophilic. In some embodiments, the compound (or at least one of the first visibly detectable performance reference compound and the second visibly detectable performance reference compound of the kits described herein) is not radioactive. In some embodiments, the compound (or at least one of the first visibly detectable performance reference compound and the second visibly detectable performance reference compound of the kits described herein) is not toxic. In some embodiments, the visibly detectable performance reference compound (or at least one of the first visibly detectable performance reference compound and the second visibly detectable performance reference compound of the kits described herein) emits a wavelength from about 0.01 nanometers to about 1.0 micrometers.

In some embodiments, the visibly detectable performance reference compound (or at least one of the first visibly detectable performance reference compound and the second visibly detectable performance reference compound of the kits described herein) is visibly detectable by an ultraviolet/visible light detector (e.g., in some embodiments, the compound emits a wavelength from about 10 nanometers to about 400 nanometers). In some embodiments, the compound emits a wavelength from about 250 nanometers to about 450 nanometers. In some embodiments, the visibly detectable performance reference compound (or at least one of the first visibly detectable performance reference compound and the second visibly detectable performance reference compound of the kits described herein) is visibly detectable by an infrared light detector (e.g., in some embodiments, the compound emits a wavelength from about 700 nanometers to about 1 micrometer). In some embodiments, the visibly detectable performance reference compound (or at least one of the first visibly detectable performance reference compound and the second visibly detectable performance reference compound of the kits described herein) is visible to a human eye (e.g., in some embodiments, the visibly detectable performance reference compound emits a wavelength from about 390 nanometers to about 700 nanometers).

In some embodiments, the analyte of interest is an organic molecule.

In some embodiments, the environment is a liquid, a solid, or a gas. In some embodiments, the environment is soil or sediment. In some embodiments, the environment is a biological fluid including, for example, urine, blood, and other fluids (e.g., interstitial fluids). In some embodiments, the biological fluid can be taken out of an organism (e.g., a blood sample taken from a human organism). In some embodiments, the biological fluid is within an organism.

In some embodiments, the invention provides a kit comprising a first passive sampling device comprising the first visibly detectable performance reference compound described herein and a second passive sampling device comprising the second visibly detectable performance reference compound described herein.

In still another aspect, the invention provides a method for detecting an analyte suspected to be in an environment, the analyte having an analyte octanol/water partition coefficient (K_{OW}), comprising contacting the environment for a time period with a passive sampling device comprising visibly detectable performance reference compound, the compound having a compound octanol/water partition coefficient (K_{OW}) (or compound log K_{OW}) that is between about 0.01% to about 10% of the analyte K_{OW} (or analyte log K_{OW}) and measuring an amount of compound in the passive sampling device, wherein the amount of compound in the passive sampling device is correlative with the amount of analyte in the environment.

In some embodiments, measuring the amount of compound comprises extracting the compound from the passive sampling device into a solvent and measuring the amount of compound in the solvent.

In various embodiments, the compound K_{OW} or compound log K_{OW} is between about 0.01% to about 5% of the analyte K_{OW} or the analyte log K_{OW}, or is between about 0.01% to about 1% of the analyte K_{OW} or the analyte log K_{OW}.

In still another aspect, the invention provides a method for detecting an analyte suspected to be in an environment, the analyte having an analyte octanol/water partition coefficient (K_{OW}), comprising contacting the environment for a time period with a passive sampling device comprising visibly detectable performance reference compound, the compound having a compound PSD/water partition coefficient that is between about 0.01% to about 10% of the analyte PSD/water partition coefficient and measuring an amount of compound in the passive sampling device, wherein the amount of compound in the passive sampling device is correlative with the amount of analyte in the environment.

In various embodiments, the compound PSD/water partition coefficient is between about 0.01% to about 5% of the analyte PSD/water partition coefficient or is between about 0.01% to about 1% of the PSD/water partition coefficient.

### BRIEF DESCRIPTION OF THE DRAWINGS

The foregoing features of embodiments will be more readily understood by reference to the following detailed description, taken with reference to the accompanying drawings, in which:

FIG. 1 is a bar graph showing the mean Log₁₀) of the elimination rate (kₑ) of two PRCs, namely unlabeled PCB-36 (not visibly detectable) and Sudan III (visibly detectable), extracted from passive sampling devices, with the bars depicting the elimination rate calculated from concentrations of PCB-36 and Sudan III in PSDs before and after deployment of the PSDs into the environment. Average Log₁₀ ke values for Sudan III and PCB-36 were within 14% agreement and not statistically different (P=0.08), indicating that Sudan III was eliminating at the same rate of PCB-36.

FIG. 2 is a line graph showing the equation

Log₁₀ kₑ=−0.59×Log₁₀ K_{PE}−1.9

plotted as Log₁₀ Kₑ (day⁻¹) versus Log₁₀ K_{PE} (L/kg/PE).

FIG. 3 is a line graph showing

Log₁₀ kₑ=−0.46×Log₁₀ K_{PE}+1.41

plotted as Log₁₀ kₑ (day-1) versus Log₁₀ K_{PE} (liters/kg/PE) for Sudan Orange G (lowest K_{PE}) with the highest Log₁₀ Kₑ Kₑ was 0.41 day⁻¹), Sudan III (intermediate K_{PE}, Kₑ was 0.046 day⁻¹), Solvent Violet 13 (intermediate K_{PE}, the Kₑ 0.16 day⁻¹), and Sudan Black B (highest K_{PE}, Kₑ was 0.00072 day⁻¹).

FIG. 4 is a line graph of a regression plot for the following equation:

Log₁₀ kₑ=−0.37×Log₁₀ K_{PE}+0.73

plotted as Log₁₀ kₑ (day-1) versus Log₁₀ K_{PE} (liters/kg/PE).

### DETAILED DESCRIPTION OF SPECIFIC EMBODIMENTS

In various aspects and embodiments, the invention provides a visibly detectable performance reference compound for use in a passive sampling device to detect an analyte of interest in an environment.

The further aspects, advantages, and embodiments of the invention are described in more detail below. The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Terms defined or used in the description and the claims shall have the meanings indicated, unless context otherwise requires. Technical and scientific terms used herein have the meaning commonly understood by one of skill in the art to which the present invention pertains, unless otherwise defined. Any conflict between an art-understood definition of a word or phrase and a definition of the word or phrase as specifically taught in this specification shall be resolved in favor of the latter. As used herein, the following terms have the meanings indicated. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

By a “passive sampling device” or “PSD” or simply a “passive sampler” is meant a device that allows passive sampling of compounds from the environment, including compounds of interest, by passive diffusion. In other words, a PSD absorbs compounds that are in the environment into which the PSD is placed.

Passive diffusion occurs when compounds move from an area of high concentration to an area of lower concentration until equilibrium conditions are reached.

A PSD, in some embodiments, absorbs only compounds that are mobile and bioavailable in the environment into which the PSD is placed. A PSD does not actively sample the environment but rather passively absorbs compounds that are present in the environment. Thus, a PSD can be constructed of any type of material that is able to absorb compounds. Such materials include, without limitation, silicone rubber, low-density polyethylene (LDPE), polyethylene (PE), polyoxymethylene (POM), polydimethylsiloxane (PDMS), polyethersulfone (PES), and polyacrylate (PA). For example, LDPE is non-porous, so it is selective and only absorbs fully dissolved and unbound molecule (e.g., free analyte). A PSD can also be a device incorporating such materials, such as Solid Phase Microextraction (SPME) fibers (USEPA, 2012) or semi-permeable membrane devices (SPMD).

In some embodiments, a passive sampler is essentially a piece of organic polymer. Non-limiting PE and POM passive samplers may be pieces of plastic sheeting that range from about 15 μm to 100 μm in thickness and can be easily cut with scissors to be as large or small as needed. For example, a polyethylene (PE) plastic drop cloth available from hardware stores can be used as passive sampler. Polyoxymethylene (POM) is a more specialized type of polymer, but it also can be purchased in large sheets and cut to size for use as a passive sampler. An SPME passive sampler can be, for example, a fiber-optic cable with an inner fiber core consisting of glass that does not readily absorb hydrophobic contaminants but the insulating polymer, polydimethylsiloxane (PDMS), coating the glass core is an absorptive material effective for passive sampling. The PDMS coating can be purchased in a variety of thicknesses from about 10 to 100 μm.

While a PSD will absorb compounds from the environment, typically a particular analyte of interest is sought to be detected and measured. By “analyte of interest” (or simply “analyte”) is any molecule or chemical that a PSD is particularly seeking out when using a PSD to analyze an environment. In some embodiments, an analyte is a hydrophobic organic chemical. Because the analyte of interest is often known in advance of placing a PSD in an environment, the material used to make the PSD can be chosen that will easily absorb the analyte of interest. For example, to detect illicit drugs (e.g., cocaine) and their metabolites at a sewage treatment plant, polar organic chemical integrative samplers (POCIS) were used as passive sampling devices (see Harman et al., Environ. Sci. Technol., 45 (13), pp 5676-5682, 2011), POCIS are designed to trap polar organic compounds from water and include a polyethersulfone (PES) membrane.

By “performance reference compound” or “PRC” is meant a chemical that is added to the passive sampling device before placement of the PSD in the environment to be analyzed. For example, the PRC may be embedded into the passive sampling device prior to placing the passive sampling device into the environment.

As used herein, by “environment” is meant any type of environment that is desired to be analyzed. The environment is sometimes referred to as an environmental media. An environment thus is any environment into which a PSD can be placed and includes, without limitation, liquid (e.g., water), gas (e.g., air), and solid environments. Thus, environments within this disclosure include, without limitation, biological fluids (e.g., blood or urine), water (e.g., surface water or water deep in a sea, ocean, river, stream, pond, or lake), aquatic sediment (e.g., the bottom of a pond or on the beach of an ocean), soil (e.g., garden soil or soil near an industrial site), and sludge. The environment can be at any temperature. For example, the environment may be very cold (e.g., −80° C. or −20° C.), may be at or just below freezing (e.g., if the environment includes snow or ice), may be at room temperature, at 37° C. (e.g., if the environment is inside a living organism such as inside a human body), or may be at a very high temperature (e.g., over 60° C. or over 100° C.).

The addition of a performance reference compound to a PSD is intended to reduce the time period needed for the PSD to be present in the environment in which the PSD is placed. After exposure to the environment, a PSD is often weighed and extracted with a solvent. The particular solvent used will depend upon the type material of the PSD and/or the type of analysis used to analyze the extract (i.e., possibly containing the analyte). The type of material of the PSD, in turn, can be selected based on the type of analytes sought to be measured.

For example, if analyte is a polycyclic aromatic hydrocarbon (PAH) or a polychlorinated biphenyl (PCB), one non-limiting polymer that may be used in a PSD is polyoxymethylene (POM). Non-limiting solvents useful for extraction from POM include hexane, methanol, or acetonitrile, or any combination of the foregoing. Another analyte that can be analyzed with a PSD composed of POM is oil. Solvents useful for extraction of oil from POM include hexane and/or acetone.

Likewise, if an analyte is a PCB, a PAH, a DDTs, a PBDE, or triclosan, one non-limiting polymer that may be used in a PSD is polyethylene (PE). Solvents useful for extraction from PE include hexane, methanol, acetonitrile, or any combination or two or more of the foregoing.

If the analyte is a PAH or a PCB, another non-limiting polymer that may be used in a PSD is silicone rubber. Non-limiting solvents useful for extraction from POM include ethyl acetate.

If the analyte is PAH, a PSD composed of polydimethylsiloxane-solid phase microextraction (PDMS-SPME) may be used, Non-limiting solvents useful for extraction of PAH from PDMS-SPME include methanol, water, or acetonitrile, or any combination of the foregoing. If the analyte is oil, and a PDMS-SPME PSD is used, a non-limiting solvent that may be used include heptane.

If the analyte is a PAH or a PCB, another non-limiting polymer that may be used in a PSD is silicone rubber. Solvents useful for extraction from POM include ethyl acetate.

The solvent (into which the PSD has been extracted) is then analyzed for organic compounds to determine the concentration of chemicals in the PSD. The concentration of chemicals that have absorbed into PSDs during an exposure to an environmental media does not directly represent the concentration of available compounds in the original environmental media. This concentration, often referred to as the freely-dissolved concentration, or C_{free} (Mayer et al., Integr. Environ. Assess. Manag. 10:197-209, 2014), is calculated from the measured steady state concentration of a compound in the sampler and a predetermined (constant) PSD-water partition coefficient (Lohmann, R., Environ. Sci. Technol. 46:606-618, 2012).

One particularly challenging issue historically with PSDs is that the time period required for the concentrations of compounds to reach steady state can be several weeks or months (Ghosh et al., Integr. Environ. Assess. Manag. 10:210-223, 2014) under most conditions in the environmental media in which PSDs are deployed. These long time periods are often not practical for several reasons, especially the increasing likelihood of loss and/or physical alteration of samplers due to theft, vandalism or other natural factors (e.g., water currents, sediment burial, organism growth), and the time constraints of environmental decision-making process, which is often on a time scale of days or weeks, not months.

PRCs thus began to be used to overcome the challenges of impractical deployment times. The use of one or more PRC enables the estimation of steady state concentrations of compounds in PSDs exposed to time periods shorter than those required to reach steady state (Huckins et al., Environ. Sci. Technol. 36:85-91, 2002; Liu et al., Environ. Sci. Technol. 47: 10104-10105, 2013; Apell and Gschwend, Environ. Sci. Technol. 48: 10301-10307, 2014; Estoppey et al., Sci. Total Environ. 499:319-326, 2014; Ghosh et al., supra). By assuming the PRCs eliminate from the PSD at the same rate that the compound(s) of interest absorbs into the PSD from the environment, the concentrations of the compound(s) of interest measured in the PSD can be “corrected” to a steady state concentration using the starting (time zero) and post-exposure concentrations of the PRC(s). This steady state concentration can be used to better calculate C_{free} in the environmental media.

Passive sampling devices are well known, with and without the use of a PRC. See, for example, US Patent Publication No. US20140069184, U.S. Pat. No. 7,059,206, PCT Patent Publication No. WO2012071629, US Patent Publication No. US20140041446, US Patent Publication No. US20110070597, Huckins et al., supra; Liu et al., supra; Apell and Gschwend, supra; Estoppey et al., supra; Ghosh et al., supra; OSWER Directive 9200, 1-110FS, U.S. Environmental Protection Agency, December 2012, all of which are incorporated herein by reference in their entireties.

Currently used PRCs are not visibly detectable and are often radioactive (e.g., by incorporating a radiolabeled) or are detectable by incorporating a stable isotope of an atom (e.g., deuterium). In other words, currently used PRCs (also referred to as “traditional PRCs”) are not visibly detectable. In some embodiments, currently used PRCs are radioactive.

For example, a traditional PRC may include stable isotope-labeled or deuterated forms of the analyte of interest (i.e., an analyte that may be contaminating the environment being analyzed). Other traditional PRCs include radiolabeled compounds with a hydrophobicity that is similar to that of the analyte of interest, or compounds that are not expected to be absorbed from the environment in significant amounts (e.g., a rare Polychlorinated Biphenyl (PCB) congeners are used as a PRC when PCBs are the analytes of interest). However, analytically pure forms of the currently used PRCs are expensive to obtain and analyze. For example, analysis of a deuterated form of Polychlorinated Biphenyl can be in the range of $500 to over $1000 per PSD, with a single PSD requiring over 30 to 60 minutes to process on typical analytical instrumentation. Additionally, since the currently used (i.e., traditional) PRCs are often toxic at low exposure levels, using these traditional PRCs in PSDs releases toxic compounds to the environment and results in potential low-level exposures to PSD sampling and analysis personnel.

Thus, by providing a visibly detectable PRC, in some embodiments, the invention provides a safer and less expensive method for analyzing the amount of compounds of interest in an environment. The invention provides a PSD comprising a visibly detectable PRC. The invention also provides kits comprising multiple visibly detectable PRCs, and kits comprising multiple PSDs, each comprising a different visibly detectable PRC.

Accordingly, in a first aspect, the invention provides a visibly detectable performance reference compound for use in a passive sampling device to detect an analyte of interest (and/or the amount of the analyte of interest) in an environment.

In another aspect, the invention provides a passive sampling device comprising a visibly detectable performance reference compound for use in a passive sampling device to detect an analyte of interest (and/or the amount of the analyte of interest) in an environment.

In some embodiments, the compound has a first octanol/water partition coefficient (K_{OW}) or first log K_{OW} and the analyte has a second K_{OW} or second log K_{OW}, wherein the first K_{OW} and the second K_{OW} are between about 0.01% to about 10% of each other, or wherein the first log K_{OW} and the second log K_{OW} are between about 0.01% to about 10% of each other. In some embodiments, the first K_{OW} and the second K_{OW} are between about 0.01% to about 5% of each other, or the first log K_{OW} and the second log K_{OW} are between about 0.01% to about 5% of each other. In some embodiments, the first K_{OW} and the second K_{OW} are between about 0.01% to about 1% of each other or the first log K_{OW} and the second log K_{OW} are between about 0.01% to about 1% of each other.

In some embodiments, the compound has a first PSD/water partition coefficient and the analyte has a second PSD/water partition coefficient, wherein the first PSD/water partition coefficient and the second PSD/water partition coefficient are between about 0.01% to about 10% of each other, or wherein the first PSD/water partition coefficient and the second PSD/water partition coefficient are between about 0.01% to about 5% of each other, or wherein the first PSD/water partition coefficient and the second PSD/water partition coefficient are between about 0.01% to about 1% of each other.

In some embodiments, the visibly detectable performance reference compound is not radioactive. In some embodiments, the visibly detectable performance reference compound is not toxic.

In another aspect, the invention provides a kit comprising a first visibly detectable performance reference compound and a second visibly detectable performance reference compound for use in a passive sampling device to detect an analyte of interest (and/or the amount of the analyte of interest) in an environment, the first compound having a first octanol/water partition coefficient (K_{OW}), the second compound having a second K_{OW}, and the analyte having a third K_{OW}, wherein the third K_{OW} is between the first K_{OW} and the second K_{OW}.

In another aspect, the invention provides a kit comprising a first visibly detectable performance reference compound and a second visibly detectable performance reference compound for use in a passive sampling device to detect an analyte of interest (and/or the amount of the analyte of interest) in an environment, the first compound having a first PSD/water partition coefficient, the second compound having a second PSD/water partition coefficient, and the analyte having a third PSD/water partition coefficient, wherein the third PSD/water partition coefficient is between the first PSD/water partition coefficient and the second PSD/water partition coefficient.

In some embodiments, the compound (or at least one of the first visibly detectable performance reference compound and the second visibly detectable performance reference compound of the kits described herein) is absorbable by a material selected from the group consisting of acetate, polyacrylate, silicone rubber, low-density polyethylene (LDPE), polyethylene (PE), polyoxymethylene (POM), polydimethylsiloxane (PDMS), polyethersulfone (PES), and/or solid phase microextraction fibers coated with such sorbents. In some embodiments, the compound (or at least one of the first visibly detectable performance reference compound and the second visibly detectable performance reference compound of the kits described herein) is absorbable by a material having an adsorption property and/or desorption property similar to a material selected from the group consisting of acetate, polyacrylate, silicone rubber, low-density polyethylene (LDPE), polyethylene (PE), polyoxymethylene (POM), polydimethylsiloxane (PDMS), polyethersulfone (PES), and/or solid phase microextraction fibers coated with such sorbents.

In some embodiments, the compound (or at least one of the first visibly detectable performance reference compound and the second visibly detectable performance reference compound of the kits described herein) is hydrophobic. In some embodiments, the compound (or at least one of the first visibly detectable performance reference compound and the second visibly detectable performance reference compound of the kits described herein) is hydrophilic. In some embodiments, the compound (or at least one of the first visibly detectable performance reference compound and the second visibly detectable performance reference compound of the kits described herein) is not radioactive. In some embodiments, the compound (or at least one of the first visibly detectable performance reference compound and the second visibly detectable performance reference compound of the kits described herein) is not toxic.

In some embodiments, the compound (or at least one of the first visibly detectable performance reference compound and the second visibly detectable performance reference compound of the kits described herein) is visibly detectable by an ultraviolet/visible light detector (e.g., in some embodiments, the compound emits a wavelength from about 10 nanometers to about 400 nanometers). In some embodiments, the compound (or at least one of the first visibly detectable performance reference compound and the second visibly detectable performance reference compound of the kits described herein) is visibly detectable by an infrared light detector (e.g., in some embodiments, the compound emits a wavelength from about 700 nanometers to about 1 micrometer. In some embodiments, the compound (or at least one of the first visibly detectable performance reference compound and the second visibly detectable performance reference compound of the kits described herein) is visible to a human eye (e.g., in some embodiments, the compound emits a wavelength from about 390 nanometers to about 700 nanometers),

In some embodiments, the analyte of interest is an organic molecule.

In some embodiments, the environment is a liquid, a solid, or a gas. In some embodiments, the environment is soil or sediment. In some embodiments, the environment is a biological fluid including, for example, urine, blood, and other fluids (e.g., interstitial fluids). In some embodiments, the biological fluid can be taken out of an organism (e.g., a blood sample taken from a human organism). In some embodiments, the biological fluid is within an organism.

In some embodiments, the invention provides a kit comprising a first passive sampling device comprising the first visibly detectable performance reference compound described herein and a second passive sampling device comprising the second visibly detectable performance reference compound described herein.

In still another aspect, the invention provides a method for detecting an analyte suspected to be in an environment, the analyte having an analyte octanol/water partition coefficient (K_{OW}) and an analyte PSD/water partition coefficient, comprising contacting the environment for a time period with a passive sampling device comprising visibly detectable performance reference compound, the compound having a compound property selected from the group consisting of a compound octanol/water partition coefficient (K_{OW}), a compound log K_{OW}, and a compound PSD/water partition coefficient that is between about 0.01% to about 10% of a property of the analyte, the analyte property selected from the group consisting of an analyte K_{OW}, an analyte log K_{OW} or an analyte PSD/water partition coefficient; and, after the time period, measuring an amount of compound in the passive sampling device, wherein the amount of compound in the passive sampling device is correlative with the amount of analyte in the environment. In various embodiments, the compound property is between about 0.01% to about 5% of the analyte property.

In a further aspect, the invention provides a method for detecting an analyte suspected to be in an environment, the analyte having an analyte octanol/water partition coefficient (K_{OW}), comprising contacting the environment for a time period with a passive sampling device comprising visibly detectable performance reference compound, the compound having a compound octanol/water partition coefficient (K_{OW}) (or compound log K_{OW}) that is between about 0.01% to about 10% of the analyte K_{OW} (or analyte log K_{OW}) or having a compound PSD/water partition coefficient that is between about 0.01% to about 10% of the analyte PSD/water partition coefficient and measuring an amount of compound in the passive sampling device, wherein the amount of compound in the passive sampling device is correlative with the amount of analyte in the environment.

In some embodiments of the methods described herein, measuring the amount of compound comprises extracting the compound from the passive sampling device into a solvent and measuring the amount of compound in the solvent.

In various embodiments, the compound K_{OW} or compound log K_{OW} is between about 0.01% to about 5% of the analyte K_{OW} or analyte log K_{OW} or is between about 0.01% to about 1% of the analyte K_{OW} analyte log K_{OW}.

By “visibly detectable” is meant that the presence of a compound (e.g., a PRC) (and, in some embodiments, its amount) can be detected by its ability to be detected by optical properties either by a human eye or by an instrument, such as spectroscopy, photometer or luminometer. In some embodiments, the visibly detectable compound emits a detectable property (e.g., color) that falls within the ultraviolet to infrared spectrum (e.g., approximately 10 nanometers to 1 micrometer). In some embodiments, the visibly detectable compound (e.g., a visibly detectable PRC) emits a detectable property (e.g., color) that falls within the spectrum visible to a human eye (e.g., approximately 390 nanometers to 700 nanometers. In some embodiments, the visibly detectable compound emits a detectable property (e.g., color) that falls within the X-radiation spectrum (e.g., approximately 0.01 nanometers to 10 nanometers). In some embodiments, the visibly detectable compound (e.g., a visibly detectable PRC) is not radioactive.

Instruments may be able to detect concentrations of the compounds in solutions that may or may not be visible to the naked eye via spectra in the visible range (approximately 390 nanometers to 700 nanometers) or ultraviolet range (approximately 10 nanometers to 400 nanometers). Many compounds can be detected in the 10 to 700 nanometer range using instrumentation and/or visible to the naked eye in solutions when present in sufficient amounts or concentrations. In some embodiments, the compound emits a wavelength from about 250 nanometers to about 450 nanometers. In this application, the use of visibly detectable compounds consists of uses of compounds in PSDs in sufficient quantity to produce a PSD that is visibly colored by the PRC and/or, when extracted, produces a solution that can be used to quantify concentration of the PRC using a nondestructive technique such as visible light/UV spectroscopy, photometer, or luminometer.

Use of a visibly detectable PRC in a PSD will allow testing to be done faster, more safely, at a cost that is much less expensive than use of a PRC that is not visibly detectable.

Furthermore, because visibly detectable PRCs are less toxic than the PRCs that are not visibly detectable and, in some embodiments, are not radioactive (as compared to radiolabeled PRCs), they are safer both for the environment into which the visibly detectable PRC-containing PSD is placed, but also to the workers handling the PSD to analyze their results. Measuring the concentration of visibly detectable PRC in the visibly detectable PRC-containing PSD also requires fewer and lower volumes of hazardous solvents and generates lower volumes and amounts of hazardous waste materials. Additionally, because the measurement of the concentration of visibly detectable PRC in solvent extracts from PSD via UV light/Visible light/Infrared light reading instruments is non-destructive, the PSD extract (e.g., PSD in a solvent) can re-measured at a later time, or even re-used. Indeed, the visibly detectable PRC in the PSD itself enables direct visual observation of passive sampling processes or results.

Measuring a traditional PRC that is not visibly detectable, or is not visibly detectable in extracts of PSDs using nondestructive visible light/UV spectroscopy, photometer, or luminometer techniques, often requires analysis on expensive, non-portable equipment, such as a high performance liquid chromatography (HPLC) instrument or a gas chromatograph. This analysis can take 30-60 minutes or more and is generally restricted to laboratory use.

In contrast, measurement of the concentration of a visibly detectable PRC in the PSD via UV light/Visible light/Infrared light reading instruments can be done in seconds. Furthermore, the visibly detectable PRC can be added and extracted from PSDs in amounts that can be easily measured via Ultraviolent/Visible/Infrared (UV/VIS/IR) spectroscopy or other visible/colorimetric measuring instruments. These instruments are fairly inexpensive to obtain and operate, and are also easily transportable for use in the field. Indeed, some are available as battery-operated handheld versions. A hand-held visible and UV light meter is sold by Omega Engineering, Inc., Stamford, Conn., USA.

Overall, using visibly detectable compounds such as dyes as PRCs in PSDs will greatly reduce the costs and analytical time requirements associated with passive sampling of sediment, soil, and water and result in increased safety for scientists using PSDs and the environmental media to which PSDs are deployed. In turn, these advantages would further facilitate the application of passive sampling to improve environmental decision making.

In some embodiments, the visibly detectable compound emits a detectable property (e.g., color) that falls within the ultraviolet to infrared spectrum (e.g., approximately 10 nanometers to 1 micrometer).

In some embodiments, the visibly detectable compound emits a detectable property (e.g., color) that falls within the infrared spectrum (e.g., approximately 700 nanometers to 1 micrometer).

In some embodiments, the visibly detectable compound emits a detectable property (e.g., color) that falls within the ultraviolet spectrum (e.g., approximately 10 nanometers to 400 nanometers). The visibly detectable PRC can thus be detected using UV spectrometry, for example.

In some embodiments, the visibly detectable compound emits a detectable property (e.g., color) that falls within the X-radiation spectrum (e.g., approximately 0.01 nanometers to 10 nanometers).

In some embodiments, the visibly detectable compound is fluorescent and emits an optical property that is visibly detectable. In some embodiments, the visibly detectable compound is luminescent and emits an optical property that is visibly detectable.

In some embodiments, a visibly detectable PRC has the similar hydrophobicity or hydrophilicity as the analyte of interest.

For example, if the analyte of interest is hydrophobic, an environment may be analyzed for the presence of the analyte of interest by placing into that environment a PSD comprising a hydrophobic visibly detectable PRC. For example, polychlorinated biphenyl (PCB) compounds are hydrophobic.

For example, if the analyte of interest is hydrophilic, an environment may be analyzed for the presence of the analyte of interest by placing into that environment a PSD comprising a hydrophilic visibly detectable PRC. For example, many pharmaceutical drugs (including hydrocortisone, paracetamol, and cocaine) are hydrophilic.

One non-limiting method for identifying a visibly detectable PRC that can be used with a passive sampling device is to use a visibly detectable PRC that has an octanol/water partition coefficient similar to the octanol/water partition coefficient of the analyte of interest. The octanol/water partition coefficient (K_{OW}) is defined as the ratio of a compound's concentration in the octanol phase to its concentration in the aqueous phase of a two-phase octanol/water system.

K_{OW}=Concentration in octanol phase/Concentration in aqueous phase

K_{OW} has become a key parameter in studies of the environmental fate of organic chemicals. A compound's K_{OW} value has been found to be related to that compound's water solubility, soil/sediment adsorption coefficients, and bioconcentration factors for aquatic life. Values of K_{OW} are usually measured at room temperature (20 or 25° C.), although the effect of temperature on K_{OW} is low—usually on the order of 0.001 to 0.01 log K_{OW} units per degree.

A compound that preferentially resides in the aqueous phase is hydrophilic whereas a compound that preferentially resides in the octanol phase is hydrophobic (or lipophilic). Therefore, compounds with low K_{OW} values (e.g., less than 10, with a log K_{OW} of less than 1.0) may be considered relatively hydrophilic. Conversely, chemicals with high K_{OW} values (e.g., greater than 10⁴) are very hydrophobic.

K_{OW} values are often expressed as the log of the value, as in log K_{OW}. Log K_{OW} are generally inversely related to water solubility and directly proportional to molecular weight of a substance. Compounds with high log K_{OW} values tend to adsorb more readily to organic matter in soils or sediments because of their low affinity for water. Compounds with very high Log K_{OW} values (e.g., a log K_{OW}>4.5) are of concern because they may have high potential to bio-concentrate in living organisms.

The log K_{OW} values of various common dyes are known, and can be readily determined for any particular dye. Table 1 below lists the log K_{OW} values for a number of common dyes.

TABLE 1

| | | Log K_{OW} value |
|---|---|---|
| Dye name | Chemical Formula | (may be estimate) |
| Citrus Red 2 | C₁₈H₁₆N₂O₃ | 5.67 |
| Coumarin dye | (2H-10benzopyran-2-one | 1.51 |
| Indigo dye | C₁₆H₁₀N₂O₂ | 3.7 |
| Phenol red | C₁₉H₁₄O₅S | 3.02 |

In some embodiments, the K_{OW} or the log K_{OW} of the visibly detectable PRC and the K_{OW} of the analyte of interest are within 10% of each other. In other words, if the log K_{OW} of an analyte of interest is 6.0, the log K_{OW} of the visibly detectable PRC is between about 5.6 and about 6.6.

Note that the analyte of interest in the environment may not be known at the time the PSD comprising the visibly detectable PRC is placed in the environment (e.g., contacting the environment with the PSD comprising the visibly detectable PRC). For example, in the soil adjacent to the wall of an abandoned compounding facility that used to a variety of pharmaceutical drugs, the analyte of interest is unknown, and there may be more than one analyte of interest. In this scenario, multiple PSDs, each comprising a different visibly detectable PRC with a different K_{OW} (octanol-water partition coefficient) or Log K_{OW}, such that each PSD has a different PSD-water partition coefficient (or Log PSD-water partition coefficient), in order to (a) identity the presence of an analyte of interest having a K_{OW} or PSD-water partition coefficient similar to (e.g., within 10% of) one of the visibly detectable PRCs in the environment (i.e., the soil) and (b) allow for fine-tuning of a particular visibly detectable PRC in the same test or a subsequent test to identify the amount of the analyte of interest in the environment being tested.

The following examples are provided to illustrate, but not limit, the invention described herein.

### Example 1

A non-limiting example of a dye that can be used as a PRC is Sudan III (CAS number: 85-86-9). Sudan III is a relatively hydrophobic organic compound with a Log octanol-water partition coefficient (Log K_{OW}) of approximately 5.7. Low K_{OW} values can be found via a variety of sources in the scientific literature for compounds for which Log K_{OW} has been measured. Alternately, one may go to a website (e.g., http://www.chemspider.com/) and enter in the name of the compound to obtain empirical or model-predicted Log K_{OW} values. In the case of Sudan III, empirical values for Log K_{OW} are not readily available, but the website provides estimates of Log K_{OW} that have been calculated by models such as the ACD/Labs Percepta model. In the case of Sudan III, the ACD/Labs Percepta model predicts a Log K_{OW} of 5.7.

The next step is the estimation of the PSD-water partition coefficient (K_{PSD}) of Sudan III. K_{PSD} is the concentration of a compound in a PSD divided by the concentration in a solution when the PSD and water are at steady state. K_{PSD} can be measured by taking a small piece of PSD and placing it in a volume of water containing a known concentration of a compound, allowing the PSD to absorb the compound during a time period long enough to attain steady state, and then measuring the concentration of the compound in the water and in the PSD. In the case of Sudan III, the K_{PSD} values have not been empirically measured. However, if the PSD is composed of polyethylene (PE), the K_{PSD}, which is a polyethylene-water partition coefficient (K_{PE}) can be estimated from the Log K_{OW} of Sudan III according to an equation presented in Ghosh et al., supra:

K_{PE}=10^{(1.22×Log KOW)-1.36}

Given a Log K_{OW} of 5.7, the predicted K_{PE} value of Sudan III is 390,000 L/kg PE.

Both hydrophobicity (as measured by Log K_{OW}) and the PSD-water partition coefficients (as measured by Log K_{PSD}) for compounds are directly related to absorption/desorption kinetics in PSDs (see Lohmann, R., supra; Ghosh et al., supra). Thus, it can be inferred that Sudan III will eliminate from a PSD deployed in sediment, water, or soil slurry at approximately the same rate as organic compounds with a similar Log K_{OW} or Log K_{PSD}. Note that the Log K_{PSD} is often referred to by the Log K of the material that the PSD is composed of. For example, a Log K_{PSD} of a PSD composed of polyethylene may be referred to as a Log K_{PE}. Similarly, a Log K_{PSD} of a PSD composed of PDMS may be referred to as a Log K_{PDMS}.

For example, with a PSD composed of PE, Sudan III would be expected to behave in the same manner as PCB-36, which also exhibits a K_{PE} of 390,000 L/kg PE (Smedes et al., Environ. Sci. Technol. 43:7047-7054, 2009). Assuming the K_{PE} values are roughly similar (i.e., within 10%) between the dye and the analyte of interest, the dye could also be an appropriate PRC to use for other compounds. For example, given that K_{PE} Sudan III K_{PE} is 390,000 L/kg, Sudan III could be used as a PRC for compounds with K_{PE} values from 350,000 L/kg to 430,000 L/kg. The following PCBs have K_{PE} values in this 350,000 L/kg to 430,000 L/kg range, according to Smedes et al., supra: PCB-35, PCB-36, PCB-37, PCB-38, PCB-39, PCB-40, PCB-41, PCB-42, PCB-43, PCB-44, PCB-47, PCB-48, PCB-49, PCB-52, PCB-59, PCB-62, PCB-64, PCB-65, PCB-69, PCB-71, PCB-73, and PCB-75.

To demonstrate this approach, an experiment was conducted using 4×10 cm pieces of PE as the active sorbent in PSDs. Twenty PSDs were created (each consisting of a 4×10 cm piece of PE). Ten of the PSDs were spiked with PCB-36 (which is not visibly detectable) and ten of the PSDs were spiked with Sudan III, a visibly detectable PRC within the scope of the present disclosure. The 20 spiked PSDs then were inserted in the top 10 cm of aquatic sediment at a marine (ocean) site by scuba divers at ten locations within a half-acre area. At each of the 10 locations, one PSD containing PCB-36 and one PSD containing Sudan III were deployed.

After 14 days, the PSDs were collected by divers, brought to the surface, placed in a shipping container, and sent to a laboratory, where they were wiped with moistened tissues, placed in small vials, and extracted with solvent. PSDs submitted for PCB analysis were extracted in a 1:1 aliquot (e.g., 50 mL) of a solvent of methylene chloride and hexane, and PSDs submitted for Sudan III analysis were extracted in 10 mL hexane solvent. Three PSDs containing Sudan III that were not deployed (i.e., were not placed in the sediment, but retained in storage) were also extracted in the same manner, and three PSDs containing PCB-36 that were not deployed (i.e., were not placed in the sediment, but retained in storage) were also extracted in the same manner. These 6 samples are referred to as “trip blanks”. The 13 solvents (from extraction of 10 exposed PSDs and 3 trip blanks PSDs) into which the PCB-36-spiked PSDs were extracted were then measured for PCBs using high-resolution gas chromatography/high-resolution mass spectrometry with an approximate 30- to 60-minute analysis time per sample. Via this analysis, the concentrations of PCB-36 in the 13 PCB-36-spiked PSDs were determined. The 13 solvents (from extraction of 10 exposed PSDs and 3 trip blanks PSDs) into which the Sudan III-spiked PSDs were extracted were measured for Sudan III on a benchtop UV/Vis spectrometer set to a wavelength of 507 nm, with an analysis time of approximately 1-2 minutes per sample. Via this analysis, the concentration of Sudan III in the 13 Sudan III-spiked PSDs was determined.

The average concentration of PCB-36 in the three trip blank PSDs was 200 ng PCB-36/g PE, and is assumed to represent the concentration of PCB-36 in the PSDs before deployment in the environment, referred to as the time zero (t=0) concentration. Concentrations of PCB-36 in the 10 PSDs exposed to the sediment for 14 days ranged from 31 to 160 ng PCB-36/g PE, referred to as the time t concentration. The time t concentration of any PRC such as PCB-36 in the individual PSDs varies due to the sampling conditions where the sampler is placed. For example, in locations where groundwater or surface water may be more forcefully interacting with the sediment where the PSD has been placed, the agitation of the sediment and sediment porewater surrounding the sampler may result in the sampler reaching steady state more quickly than a PSD that is planted in very tight, stagnant sediment. A PSD in the former condition will eliminate PRCs such as PCB-36 more quickly, which will result in a lower time t concentration than PSDs in the latter (stagnant) conditions. It could be said that the “sampling rate” at which compounds are taken up (or eliminated from) the PSD vary.

The “sampling rate” of the PSD is expressed numerically as a term referred to as the elimination rate (kₑ). kₑ values can be calculated using the following equation (Lohmann, supra):

$$PRC {k}_{e} = ln ( \frac{[ {PSD}_{t = 0} ]}{[ {PSD}_{t} ]} ) \div Time$$

where:
PSDₜ=the concentration of the PRC in the PSD after the deployment (obtained from the PSD sampler exposed in the environment),
PSDₜ₌₀=the average concentration of the PRC in the PSD at the beginning of the exposure (obtained from measurement of the PSDs that were not deployed in the environment), and
Time=Deployment time.

kₑ values can be calculated each PRC for each PSD. For example, the time t concentration of PCB-36 in the PCB-36-spiked PSD deployed at Station 6 (one of the ten locations at which PSDs were deployed) was 110 ng PCB-36/g PE. Given the average t=0 concentration of 200 ng PCB-36/g PE for the unexposed PCB-36-spiked PSDs and a deployment time of 14 days, the above equation indicates a kₑ for PCB-36 in this PSD of 0.043 day-1.

The same approach can be used to estimate kₑ values for Sudan III using the data from the 10 Sudan III-spiked PSDs deployed in sediment and the 3 trip blank Sudan III-spiked PSDs. The average concentration of Sudan III in the three trip blank PSDs was 32 mg Sudan III/g PE, and is assumed to represent the t=0 concentration of Sudan III in the PSDs that were deployed in the sediment. Concentrations of Sudan III in the 10 PSDs exposed to the sediment for 14 days (time t concentration) ranged from 16 to 21 mg Sudan III/g PE. As with PCB-36, the time t concentration of Sudan III in the individual PSDs varies due to the sampling conditions where the sampler is placed. For example, the time t concentration of Sudan III in the Sudan III-spiked PSD deployed at Station 6 (deployed within approximately a meter of the PCB-36-spiked PSD that was deployed at Station 6) was 21 mg Sudan III/g PE. Given the average t=0 concentration of 32 mg Sudan III/g PE for the unexposed Sudan III-spiked PSDs and a deployment time of 14 days, the above equation indicates a kₑ for Sudan III in this PSD of 0.032 day⁻¹. The kₑ values for the PCB-36- and Sudan III-spiked PSDs deployed at Station 6 (0.043 and 0.032 day⁻¹, respectively) are similar (within a 25% difference), indicating that Sudan III is eliminating from the PSD at approximately the same rate as PCB-36. Because of this, Sudan III could be an acceptable PRC substitute for PCB-36.

For example, in the PCB-36-spiked PSD deployed at Station 6, the PCB analysis indicated that a detectable amount of PCB-35 absorbed from the sediment into the PSD during the 14 days it was exposed to the sediment. The time t concentration of PCB-35 was found to be 0.14 ng PCB-35/g PE. PCB-35 and PCB-36 have very similar hydrophobicities, and, not surprisingly, have the same K_{PE} of 390,000 L/kg PE (Smedes et al., supra). Because of this, the kinetics of depuration and absorption for these two chemicals can be assumed to be approximately equal. Thus, the kₑ value of the PCB-36 (the PCB PRC) can be used to estimate the steady state concentration of PCB-35 (i.e., the concentration of PCB-35 that would have eventually been attained in this PSD if the PSD had been left in the sediment at Station 6 for additional time). The general equation to estimate the steady state concentration of an analyte using a kₑ value and a measured pre-steady state concentration of an analyte is (Lohmann, supra):

$$[ {Analyte}_{Steady State} ] = \frac{[ {Analyte}_{t} ]}{1 - {e}^{- {k}_{e} \times Time}}$$

where:
Analyte_{Steady State}=the concentration of the analyte of interest in the PSD at steady state,
Analyteₜ=the time t concentration of the analyte of interest in the PSD after the deployment (obtained from the PSD sampler exposed in the environment),
kₑ=the elimination rate for the analyte of interest, from measurement of the PSDs that were not deployed in the environment)
Time=Deployment time.

Given a time t concentration of PCB-35 of 0.14 ng PCB-35/g PE, an assumed kₑ of 0.043 day⁻¹ (calculated using PCB-36 data as described above) and a deployment time of 14 days, the equation predicts a steady state concentration of PCB-35 of 0.31 ng PCB-35/g PE. This value can then be used in a variety of environmental data evaluations, such as calculation of the dissolved concentrations of PCB-35 in the sediment at Station 6 or prediction of the concentration of PCB-35 in organisms living in sediment at Station 6.

The kₑ value derived from the Sudan III-spiked PSD at Station 6 can also be used in the above approach to calculate the steady state concentration of PCB-35 in the PSD deployed at Station 6. Given a time t concentration of PCB-35 of 0.14 ng PCB-35/g PE, an assumed kₑ of 0.032 day⁻¹ (calculated using Sudan III data as described above) and a deployment time of 14 days, the equation predicts a steady state concentration of PCB-35 of 0.39 ng PCB-35/g PE. This steady state concentration of PCB-35 is within approximately 20% of the estimated steady state concentration of PCB-35 estimated when using the PCB-36 PRC data (e.g., 0.31 ng PCB-35/g PE), indicating that Sudan III could replace PCB-36 as a PRC.

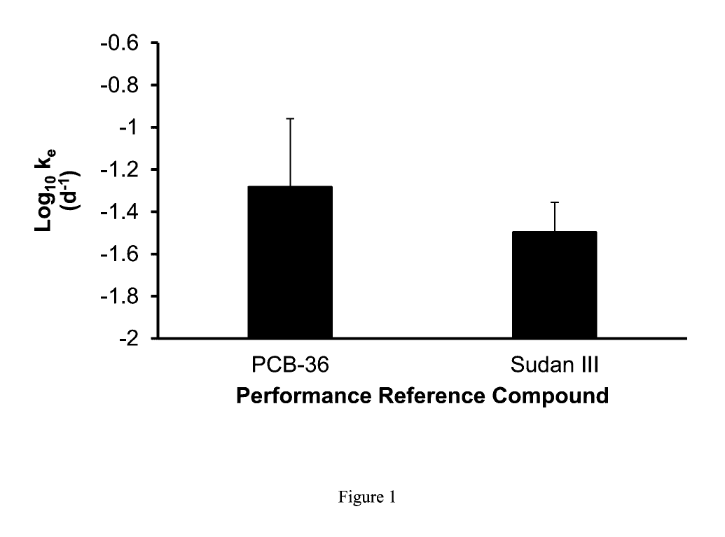

Calculation of kₑ values can be conducted using the time t concentration of Sudan III in the Sudan III-spiked PSDs deployed in sediment at the other 9 locations and the time t concentration of PCB-36 in the PCB-36-spiked PSDs deployed in sediment at the other 9 locations. FIG. 1 is a bar graph showing the mean Log₁₀ of the elimination rate kₑ of PCB-36 and Sudan III (ke values were Log₁₀-transformed due to the wide variation in kₑ values among the 10 stations). As can be seen from FIG. 1, average Log₁₀ kₑ values for Sudan III and PCB-36 were within 14% agreement and not statistically different (P=0.08), indicating that Sudan III was eliminating at the same rate of PCB-36, as would be predicted given the similarity in the two compounds' hydrophobicities/PSD-water partition coefficients.

### Example 2

If multiple dyes (i.e., visibly detectable PRCs) that exhibit a range of hydrophobicities and/or PSD-water partition coefficients are used in a passive sampler (or multiple samplers in the same media), a relationship between the amount of each PRC eliminated during deployment can be used to develop a model that can be used to correct the measured concentrations of analytes of interest to steady state concentrations in the same approach as that used in other applications of traditional PRCs (e.g., Liu et al., supra; Apell and Gschwend, supra; Estoppey et al., supra). Several additional dyes compounds are listed below in Table 2, and exhibit hydrophobicities (as measured by Log K_{OW}) that indicate their potential use as PRCs in measuring a broad range of organic compounds via passive sampling, including PCBs, Polycyclic Aromatic Hydrocarbons (PAHs), pesticides, and dioxins/furans. If multiple dyes as PRCs in this manner, traditional PRCs would not be needed.

For example, it could be demonstrated that multiple dyes could be used as PRCs for analyte compounds that are of different hydrophobicities than the dyes used as PRCs. Some non-limiting dyes are provided in Table 2

Table 2.

Additional dye compounds potentially useful as PRCs in passive sampling applications, listing their CAS numbers for reference (based on the CAS Registry number, assigned by the Chemical Abstracts Service of the American Chemical Society, Columbus, Ohio, USA).

TABLE 2

| Dye Compound | CAS Number | Log K_{OW} |
|---|---|---|
| Sudan III | 85-86-9 | 5.7 |
| Solvent Violet 13 | 81-48-1 | 6.2 |
| Sudan I | 842-07-9 | 5.5 |
| Sudan Orange G | 2051-85-6 | 3.9 |
| Rhodamine B | 81-88-9 | 1.8 |

To demonstrate this approach, an experiment could be conducted using two 4×10 cm pieces of PE as the active sorbent in PSDs. Two PSD could be created (each consisting of a 4×10 cm piece of PE). Both PSDs could be spiked with three dye PRCs: Sudan III, Solvent Violet 13, and Sudan Orange G (see Table 2 above). These additional dyes were selected according to their hydrophobicity relative to that of Sudan III (Log K_{OW} of 5.7); Solvent Violet 13 is more hydrophobic (Log K_{OW} of 6.2) and Sudan Orange G is less hydrophobic (Log K_{OW} of 3.9). See Table 2.

Using the equation described in Example 1 to predict the K_{PE} value of a compound using its Log K_{OW}, the K_{PE} values of Sudan Orange G, Sudan III, Solvent Violet 13 would be 2,500, 390,000, and 1,600,000 L/kg PE, respectively. PCB-60 is an analyte of interest that may occur in the environment, with a K_{PE} value of 710,000 L/kg (Smedes et al. supra). The K_{PE} values of the 3 dyes are dissimilar to that of PCB-60, yet they may be used to provide a kₑ for PCB-60 according to the following approach. One PSD containing the three above dyes would be deployed into sediment for a period of 28 days, and the other PSD containing the three above dyes would be held in storage. Following retrieval of the PSD deployed in the sediment, both PSDs would be extracted in solvent (e.g., 50-mL mixture of hexane and methylene chloride). The two solvents would then be analyzed on a benchtop UV/Vis spectrophotometer at wavelengths of 388 nm (to indicate the concentration of Sudan Orange G in the solvent), 507 nm (to indicate the concentration of Sudan III in the solvent), and 582 nm (to indicate the concentration of Solvent Violet 13 in the solvent). Concentrations of these dyes in the PE could be calculated assuming a known mass of each PE piece. The solvent extract of the PSD that had been exposed to the sediment for 28 days would be measured for PCBs using high-resolution gas chromatography/high-resolution mass spectrometry with an approximate 30- to 60-minute analysis time per sample. Via this analysis, the concentration of PCB-60 in the PSD that had been exposed to the sediment for 28 days could be determined.

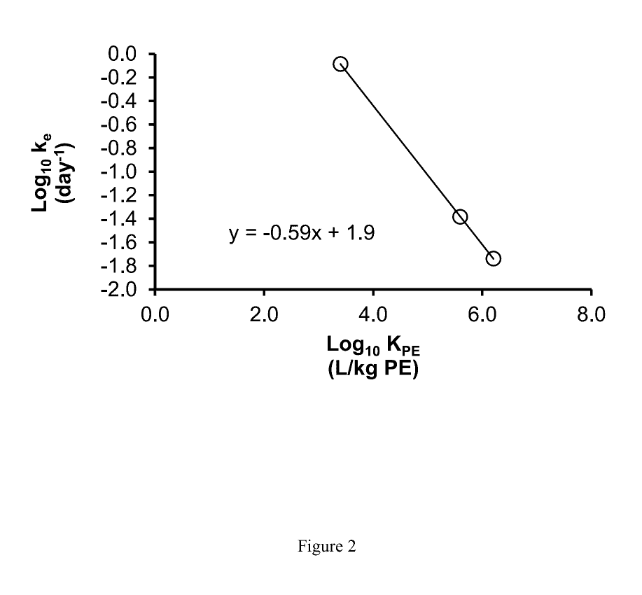

Using the concentrations of the three dyes in the unexposed PSD (to represent the t=0 concentration of each dye) and the concentration of the three dyes in the PSD that had been exposed to the sediment for 28 days (the time t concentrations), one could estimate the kₑ values for the three dyes using the equation provided in Example 1. The kₑ values would be expected to be indirectly related to K_{PE}: the kₑ for Sudan Orange G (lowest K_{PE}) would be high (e.g., hypothetically 0.8 day⁻¹), the kₑ for Sudan III (intermediate K_{PE}) would be moderate (e.g., hypothetically 0.04 day⁻¹), and the kₑ for Solvent Violet 13 (highest K_{PE}) would be low (e.g., hypothetically 0.02 day⁻¹). These kₑ values would be Log₁₀-transformed (e.g., to values of −0.1, −1.4, and -1.7 day⁻¹). The K_{PE} values for the three compounds would also be Log₁₀-transformed to 3.4, 5.6, and 6.2 L/kg PE. A linear regression model would then be estimated using the Log₁₀ kₑ values as the dependent variable (y axis) and the Log₁₀ K_{PE} values as the independent variable (y axis). The hypothetical regression plot is shown in FIG. 2, and indicates that Log₁₀ kₑ could be predicted by the following equation:

Log₁₀ kₑ=−0.59×Log₁₀ K_{PE}−1.9

FIG. 2 is a line graph showing this equation plotted as Log₁₀ Ke (day-1) versus Log₁₀ K_{PE} (liters/kg/PE). The K_{PE} of PCB-60 is 710,000 liters/kg (and the Log₁₀ K_{PE} is 5.9 L/kg PE). By using the equation from the plot shown in FIG. 2, the predicted kₑ value of PCB-60 could be interpolated from its known K_{PE} value. The estimated hypothetical value for PCB-60 would be 0.028 day⁻¹. If the measured concentration (time t concentration) of the PCB-60 in the PSD exposed to the sediment for 28 days was found to be hypothetically 1 ng PCB-60/g PE, the estimated PCB-60 kₑ value of 0.028 day⁻¹ could be used in the equation shown in Example 1 to estimate the steady state concentration of PCB-60 in the PSD.

$$[ {Analyte}_{Steady State} ] = \frac{[ {Analyte}_{t} ]}{1 - {e}^{- {k}_{e} \times Time}}$$

The equation would be as follows:

$$[ {Analyte}_{Steady State} ] = \frac{[ 1 ]}{1 - {e}^{- 0.028 \times 28}} ,$$

And therefore,

[Analyte_{Steady State}]=1.8

As shown above, the hypothetical result of this calculation would be 1.8 ng PCB-60/g PE. This steady state concentration could then be used in a variety of environmental data evaluations, such as calculation of the dissolved concentrations of PCB-60 in the sediment to which the PSD was exposed or prediction of the concentration of PCB-60 in organisms living in which the PSD was exposed.

### Example 3

This example describe how the Sudan III dye will be used as a visibly detectable PRC in a PSD to detect and/or measure the amount of polychlorinated biphenyl analytes in the soil lining the banks of the Charles River in Cambridge, Mass., USA.

Sheets of polyethylene (PE) are cut into multiple rectangles of the same size (e.g., 4×10 cm), placed in protective mesh envelopes, and cleaned with solvents including, for example, methylene chloride, hexane, toluene, and the like, to remove any trace organic compounds (PCBs) that may be present.

The PSDs are then divided into groups, with each group containing at least three PSDs. The first group will be untreated. The second group will be soaked for 24 hours in Sudan III dye solution, the third group will be soaked for 24 hours in a solution containing PCB-36, and the fourth group will be soaked for 24 hours in a solution containing both PCB-36 and Sudan III dye.

One PSD from each Group (i.e., the 4×10 cm rectangle) will be extracted in solvent (e.g., hexane), and the solvent will be analyzed by gas chromatography (GC) methods to determine the amount of PCB-36 and by a visible light meter. Note that Sudan III has a color that is at 507 nanometers, which is visible to the human eye. For PCB analysis by gas chromatography (GC), the PSD will be extracted in approximately 50 mL of solvent. For Sudan III analysis by a visible light meter, the PSD will be extracted in approximately 10 mL solvent. The time to perform the GC analysis takes 60 minutes and uses $500 dollars of materials and reagents, some of which must be disposed of as hazardous waste. The time to perform the visible light meter takes 5-10 seconds and uses no additional materials or reagents.

All of the PSD rectangles of the each of the groups are then placed into the soil along the banks of the Charles River in Cambridge, Mass., USA, and the squares are left there for 30 days. Each of the PSDs is placed at the same depth and at the same distance from the water of the Charles River.

The PSDs are then collected from the environment (i.e., the Charles River) and tested by GC and by a visible light meter to detect the amount of PCB-36 and Sudan III.

The findings will show that the amount of Sudan III that has faded from the Sudan III-loaded PSDs closely correlates with the amount of PCB-36 that has eluted from the PCB-36 loaded PSDs. The PSD that was loaded with both Sudan III and PCB-36 will support this finding.

All PSDs will be found to absorb the same amount of PCBs of similar hydrophobicity to PCB-36 and Sudan III that could be expected to be found at detectable concentrations at this site (e.g., PCB-34, PCB-35, PCB-37) given the relatively common occurrence of these three congeners in standard PCB Aroclor mixtures historically used in industrial applications.

This experiment shows that use of Sudan III a visibly detectable PRC is as accurate as using deuterated PCB-36 as a more traditional PRC that is not visibly detectable.

### Example 4

This example describes how the rhodamine dye will be used as a visibly detectable PRC in a PSD to detect and/or measure the amount of methyltrexate, a chemotherapeutic drug, in a pond adjacent to a factory that manufactures methyltrexate.

The factory is suspected of dumping excess methyltrexate into the pond.

Sixteen polar organic chemical integrative samplers (POCIS) are obtained from Environmental Sampling Technologies (EST Inc., St. Joseph, Mo., USA) and divided into four groups. Group I is untreated. Group II is soaked in 3H-labeled methyltrexate, with the 3H labeled methyltrexate serving as a traditional PRC. Group III is soaked in rhodamine. Group IV is soaked in rhodamine and 3H-labeled methyltrexate.

One POCIS from each group is analyzed by visual light spectroscopy, mass spectrometry, and by a radiation detector to measure the amount of rhodamine, 3H labeled methyltrexate, and unlabeled methyltrexate.

All 16 POCIS are then submersed into the pond at the same depth and distance from the factory.

Because rhodamine dye and the methyltrexate (unlabeled and labeled) are hydrophilic, the exposure time can be quite short. Thus, after seven days, the POCIS are removed from the pond environment and analyzed by visual light spectroscopy (e.g., using the Genesys 10S UV-VIS Spectrophotometer sold by Thermo Scientific (Waltham, Mass.)), mass spectrometry, and by a radiation detector to measure the amount of rhodamine, 3H labeled methyltrexate, and unlabeled methyltrexate.

The findings are expected to show that all sixteen POCIS absorbed the same amount of unlabeled methyltrexate. The POCIS in Group III and Group IV are expected to be found to have both lost the same amount of rhodamine. Group II and Group IV are expected to found to have both lost the same amount of 3H-labeled methyltrexate.

The findings will show that the amount of rhodamine that has faded from the rhodamine-loaded POCIS in Groups III and IV closely correlates with the amount of 3H-labeled methyltrexate that has eluted from the POCIS in Groups II and IV.

This experiment shows that use of rhodamine as a visibly detectable PRC is as accurate as using 3H-labeled methyltrexate as a more traditional PRC that is not visibly detectable.

### Example 5

This example describes how the rhodamine dye will be used as a visibly detectable PRC in a PSD to detect and/or measure the amount of a methyltrexate or its breakdown products (i.e., its metabolites) in the urine of a human cancer patient.

The patient has been taking methyltrexate as a drug for one week. This study is done to determine if the methyltrexate is being metabolized (i.e., broken down into breakdown products) properly by the patient.

Sixteen polar organic chemical integrative samplers (POCIS) are obtained from Environmental Sampling Technologies (EST Inc, St. Joseph, Mo.) and divided into four groups. Group I is untreated. Group II is soaked in 3H-labeled methyltrexate, with the 3H labeled methyltrexate serving as a PRC. Group III is soaked in rhodamine. Group IV is soaked in rhodamine and 3H-labeled methyltrexate.

One POCIS from each group is analyzed by visual light spectroscopy, mass spectrometry, and/or by a radiation detector to measure the amount of rhodamine, 3H labeled methyltrexate, and unlabeled methyltrexate.

All 16 POCIS are then submersed into urine collected from the patient.

After thirty minutes, the POCIS are removed from the urine environment and placed in a high purity methanol solvent to extract the dye and/or 3H-labeled methyltrexate retained in the POCIS. The solvent used for extraction will then be analyzed by visual light spectroscopy, mass spectrometry, and by a radiation detector to measure the amount of rhodamine, 3H labeled methyltrexate, and unlabeled methyltrexate.

The findings are expected to show that all POCIS in all four Groups absorbed the same amount of unlabeled methyltrexate. The POCIS in Group III and Group IV are expected to be found to have both lost the same amount of rhodamine. The POCIS in Group II and Group IV are expected to found to have both lost the same amount of 3H-labeled methyltrexate.

The findings will show that the amount of rhodamine that has faded from the rhodamine-loaded POCIS in Groups III and IV closely correlates with the amount of 3H-labeled methyltrexate that has eluted from the POCIS in Groups II and IV.

This experiment shows that use of rhodamine as a visibly detectable PRC is as accurate as using 3H-labeled methyltrexate as a more traditional PRC that is not visibly detectable. This experiment also shows that POCIS and a visibly detectable PRC, namely rhodamine dye, can be used to detect methyltrexate and its breakdown products in human urine.

### Example 6

This example describes how the non-toxic phenol red dye, which has a log K_{OW} of 3.02, will be used as a visibly detectable PRC in a PSD to detect and/or measure the amount of methyltrexate, a chemotherapeutic drug, in a human cancer patient.

The log K_{OW} values of five well-known drugs has been recently reported (Scheytt et al., Water, Air, and Soil Pollution 165: 3-11, 2005). Table 3 lists the drugs and their reported log K_{OW} values.

TABLE 3
Drugs and log KOW values.

| Drug name | Log K_{OW} value |
|---|---|
| Carbamazepine | 1.51 |
| Clofibric acid | 2.88 |
| Diclofenac | 1.9 |
| Ibuprofen | 2.48 |
| Propyphenazone | 2.02 |

Clofibric acid is a metabolite (i.e., breakdown product) of the cholesterol-lowering pharmaceutical drug clofibrate. A patient receiving clofibrate to reduce his cholesterol will be examined to determine if he is metabolizing the clofibrate he is ingesting.

Phenol red dye, with a log K_{OW} of 3.02, will be used as a visibly detectable PRC for clofibric acid, which has a log K_{OW} of 2.88.

3.02 minus 2.88 equals 0.14. 0.14 divided by 3.02 equals 4.6%. Therefore, the log K_{OW} of the visibly detectable PRC (namely phenol red) and the analyte (namely clofibric acid) are within 5% of each other.

Eight POCIS (comprising polyethersulfone (PES) membranes) are obtained from Environmental Sampling Technologies (EST Inc., St. Joseph, Mo.). These POCIS are relatively non-toxic to human. The POCIS are divided into four groups of two each. Group I is untreated. Group II is soaked in 3H-labeled clofibric acid, with the 3H-labeled clofibric acid serving as a PRC. Group III is soaked in phenol red. Group IV is soaked in phenol red and 3H-labeled clofibric acid.

One POCIS from each group is analyzed by visual light spectroscopy, HPLC (high performance liquid chromatography), and by a radiation detector to measure the amount of rhodamine, 3H labeled clofibric acid, and unlabeled clofibric acid.

Four sub-cutaneous incisions are made onto the patient's back. The remaining four POCIS (one from each of Group I, Group II, Group III, and Group IV) are inserted into each of the incisions, and the incisions are closed with suturing. Thus, the environment in which the four POCIS are deployed is a biological fluid (e.g., blood and lymphatic fluid) and the POCIS contacts muscle, subcutaneous fat, and tissue in the dermis and epidermis.

The four POCIS are left in situ in the patient for seven days.

The removed POCIS are then placed in a methanol solvent (approx. 20 mL per POCIS) to extract the phenol red dye and/or 3H-labeled clofibric acid retained in the POCIS. The solvent used for extraction will then be analyzed by visual light spectroscopy, mass spectrometry, and by a radiation detector to measure the amount of phenol red, 3H labeled methyltrexate, and unlabeled methyltrexate.

The findings are expected to show that all four POCIS absorbed the same amount of unlabeled clofibric acid from the patient. The POCIS in Group III and Group IV are expected to be found to have both lost the same amount of phenol red. Group II and Group IV are expected to found to have both lost the same amount of 3H-labeled clofibric acid.

The findings will show that the amount of phenol red that has faded from the phenol red-loaded POCIS in Groups III and IV closely correlates with the amount of 3H-labeled clofibric acid that has eluted from the POCIS in Groups II and IV.

This experiment shows that use of phenol red as a visibly detectable performance reference compound is as accurate as using 3H-labeled clofibric acid as a more traditional performance reference compound that is not visibly detectable.

### Example 7

If multiple dyes (i.e., visibly detectable PRCs) that exhibit a range of hydrophobicities and/or PSD-water partition coefficients are used in a passive sampler (or multiple samplers in the same media), a relationship between the amount of each PRC eliminated during deployment can be used to develop a model that can be used to correct the measured concentrations of analytes of interest to steady state concentrations in the same approach as that used in other applications of traditional PRCs (e.g., Liu et al., supra; Apell and Gschwend, supra; Estoppey et al., supra). Several additional dyes compounds are listed below in Table 4, and exhibit hydrophobicities (as measured by Log K_{OW}) that indicate their potential use as PRCs in measuring a broad range of organic compounds via passive sampling, including PCBs, Polycyclic Aromatic Hydrocarbons (PAHs), pesticides, and dioxins/furans. If multiple dyes are used as PRCs in this manner, traditional PRCs would not be needed.

For example, it could be demonstrated that multiple dyes could be used as PRCs for analyte compounds that are of different hydrophobicities than the dyes used as PRCs. Some non-limiting dyes are provided in Table 4

Table 4.

Additional dye compounds potentially useful as PRCs in passive sampling applications, listing their CAS numbers for reference.

TABLE 4

| Dye Compound | CAS Number | Log K_{OW} |
|---|---|---|
| Sudan III | 85-86-9 | 5.7 |
| Solvent Violet 13 | 81-48-1 | 6.2 |
| Sudan Black B | 4197-25-5 | 8.8 |
| Sudan Orange G | 2051-85-6 | 3.9 |

To demonstrate this approach, an experiment was conducted using ten 3×3 cm pieces of PE as the active sorbent in PSDs. Eight of the PSDs were spiked with one of the four dye PRCs: Sudan III, Solvent Violet 13, Sudan Black B, and Sudan Orange G (see Table 4 above). That is, two PSDs were spiked with Sudan III, two PSDs were spiked with Solvent Violet 13, two PSDs were spiked with Sudan Black B, and two PSDs were spiked with Sudan Orange G. These additional dyes were selected according to their hydrophobicity relative to that of Sudan III (Log K_{OW} of 5.7); Solvent Violet 13 and Sudan Black B are more hydrophobic (Log K_{OW} of 6.2 and 8.8, respectively) and Sudan Orange G is less hydrophobic (Log K_{OW} of 3.9). See Table 4. Two additional 3×3 cm PSDs were spiked with 10 unlabeled PCB PRCs that are not visibly detectable, but are routinely used as PRCs in passive sampling applications.

Using the equation described in Example 1 to predict the K_{PE} value of a compound using its Log K_{OW}, the K_{PE} values of Sudan Orange G, Sudan III, Solvent Violet 13, and Sudan Black B would be 2,500, 390,000, 1,600,000, and 2,400,000,000 L/kg PE, respectively. The PCB PRCs added to the two additional PSDs include 10 PCBs with K_{PE} values that range from 130,000 to 60,000,000 L/kg (Smedes et al. supra). The range of the K_{PE} values for the PCB PRCs falls within the range of the K_{PE} values for the dyes.

To demonstrate that dyes could estimate the steady state concentration of a variety of target analytes in a manner similar to that based on PCB PRCs, four dye-spiked PSDs and one PSD containing the PCB PRCs were deployed into five 8-oz jars containing approximately 100 g (wet weight) sediment and 150 mL water. The five jars containing the PSDs were closed and agitated for a period of 14 days on a shaker table. The other four dye-spiked PSDs and PCB-spiked PSD were held in storage. Following retrieval of the PSDs deployed in the sediment after 14 days, the PSDs were extracted in solvent (e.g., 50-mL mixture of hexane and methylene chloride). The solvents from the dye-spiked PSDs were analyzed on a benchtop UV/Vis spectrophotometer at wavelengths of 380 nm (to indicate the concentration of Sudan Orange G in the solvent), 465 nm (to indicate the concentration of Sudan III in the solvent), 590 nm (to indicate the concentration of Solvent Violet 13 in the solvent), and 557 nm (to indicate the concentration of Sudan Black B in the solvent). Concentrations of these dyes in the PE could be calculated assuming a known mass of each PE piece. The solvent extracts of the PSDs that had been exposed to the sediment for 14 days and held in storage for 14 days would be measured for PCBs using high-resolution gas chromatography/high-resolution mass spectrometry with an approximate 30- to 60-minute analysis time per sample.

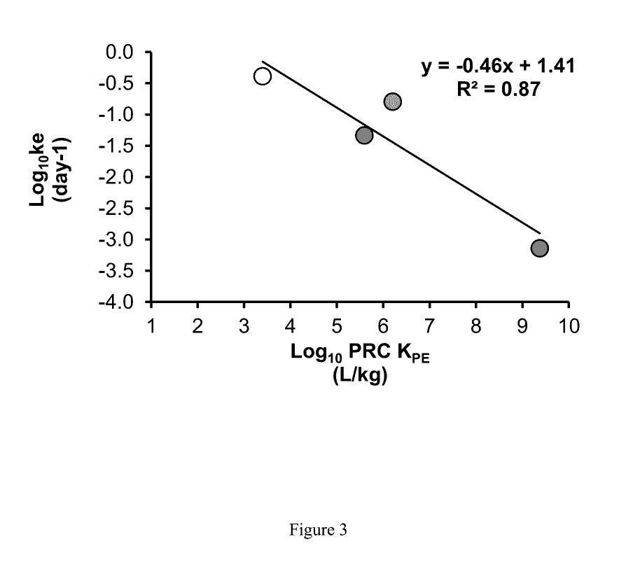

Using the concentrations of the four dyes in the four unexposed PSDs (to represent the t=0 concentration of each dye) and the concentration of the four dyes in the PSDs that had been exposed to the sediment for 14 days (the time t concentrations), one could estimate the kₑ values for the four dyes using the equation provided in Example 1. The kₑ values were indirectly related to K_{PE}: the kₑ for Sudan Orange G (lowest K_{PE}) was high (i.e., 0.41 day⁻¹), the kₑ for Sudan III (intermediate K_{PE}) was moderate (i.e., 0.046 day⁻¹), the kₑ for Solvent Violet 13 (intermediate K_{PE}) was moderate (i.e., 0.16 day⁻¹), and the kₑ for Sudan Black B (highest K_{PE}) was lowest (i.e., 0.00072 day⁻¹). These kₑ values would be Log₁₀-transformed (e.g., to values of −0.39, −1.3, −0.8, and -3.1 day⁻¹). The K_{PE} values for the four compounds would also be Log₁₀-transformed to 3.4, 5.6, 6.2, and 9.4 L/kg PE. A linear regression model would then be estimated using the Log₁₀ kₑ values as the dependent variable (y axis) and the Log₁₀ K_{PE} values as the independent variable (y axis). The regression plot of these values is shown in FIG. 3, and indicates that Log₁₀ kₑ could be predicted by the following equation:

Log₁₀ kₑ=−0.46×Log₁₀ K_{PE}+1.41

FIG. 3 is a line graph showing this equation plotted as Log₁₀ kₑ (day-1) versus Log₁₀ K_{PE} (liters/kg/PE).

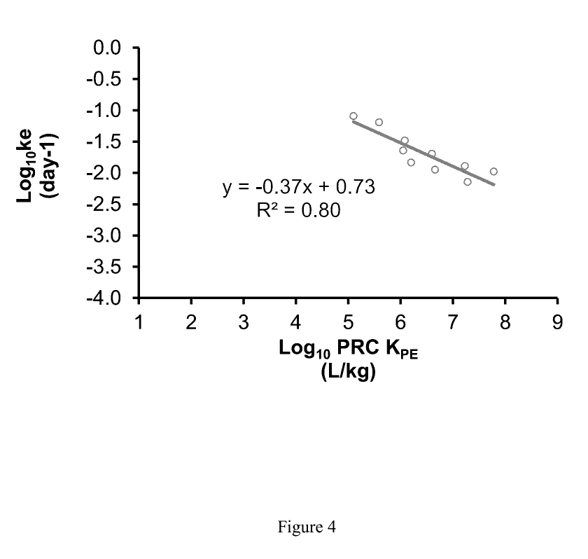

Using the concentrations of the 10 PCB PRCs in the one unexposed PSD (to represent the t=0 concentration of each PCB PRC) and the concentrations of the 10 PCB PRCs in the PSDs that had been exposed to the sediment for 14 days (the time t concentrations), one could estimate the kₑ values for the 10 PCB PRBs using the equation provided in Example 1. Using the same approach as described above for the dye PRCs, a regression plot using the PCB PRC data is shown in FIG. 4, and indicates that Log₁₀ kₑ could be predicted by the following equation:

Log₁₀ kₑ=−0.37×Log₁₀ K_{PE}+0.73

FIG. 4 is a line graph showing this equation plotted as Log₁₀ kₑ (day-1) versus Log₁₀ K_{PE} (liters/kg/PE).

Using the equations from the plots shown in FIGS. 3 and 4, the predicted kₑ values of a variety of target analytes can be interpolated using four known K_{PE} values that span the range of typical target analytes (i.e., 100,000 to 32,000,000 L/kg), as shown in Table 5.

Table 5.

kₑ values and steady state concentrations in PSDs for four hypothetical analytes, as predicted using either a dye PRC or a PCB PRC approach.

TABLE 5

| | | | | Predicted steady state concentration of a | |
|---|---|---|---|---|---|
| | | | Hypothetical concentration | target analyte in a PSD measured after a | |
| | Predicted ke (day⁻¹) | | of a target analyte in a | hypothetical 14-day deployment (ng/g) | |
| | Dye | PCB PRC | PSD measured after a | Dye | PCB PRC |
| K_{PE} (L/kg) | Approach | Approach | 14-day deployment (ng/g) | Approach | Approach |
| 100,000 | 0.13 | 0.076 | 1 | 1.2 | 1.5 |
| 1,000,000 | 0.044 | 0.032 | 1 | 2.2 | 2.7 |
| 10,000,000 | 0.015 | 0.014 | 1 | 5.2 | 5.7 |
| 32,000,000 | 0.0089 | 0.0090 | 1 | 8.5 | 8.4 |

Using the general equation (shown in Example 1) to estimate the steady state concentration of four analytes using these predicted kₑ values and hypothetical concentrations of each of the four hypothetical analytes in a PSD after a 14-day deployment in sediment agitated for a period of 14 days on a shaker table (i.e., 1 ng/g), the steady state concentrations of the hypothetical analytes in PSDs can be predicted (Table 5). For example, the steady state concentration of target analyte with a K_{PE} of 10,000,000 L/kg that was found to be at a concentration of 1 ng/g in a PSD would be 5.2 ng/g (as predicted using the dye approach and subsequent data) or 5.7 ng/g (as predicted using the PCB PRC approach and subsequent data). These values are very similar (within a factor of 1.1). In fact, for the four hypothetical analyte examples shown in Table 5, the steady state concentrations predicted by the dye and PCB PRC approaches are within a factor of 1.0 to 1.3 agreement, indicating that the dye approach provides PRC data that is similar to that of the PCB PRCs. As such, the dye PRC approach could be used in place of PCB PRCs to estimate steady state concentrations of analytes in PSDs.

### Example 8

In this example, a dye is used to quantify the increase in sampling rate that occurs due to agitation of a PSD and sediment sample. Agitation of PSDs and sediment is known to increase the sampling rate by increasing the absorption of analytes by a PSD (Ghosh et al., supra).

To demonstrate this approach, an experiment was conducted using pieces of PE as the active sorbent in PSDs. Six 3×3 cm PSDs and fourteen 4×10 cm PSDs were spiked with Sudan III dye. Eleven of the larger (4×10 cm) PSDs were inserted in the top 10 cm of aquatic sediment at a marine (ocean) site by scuba divers at ten locations within a half-acre area. Three of the smaller (3×3 cm) PSDs were deployed into five 8-oz jars containing approximately 100 g (wet weight) sediment and 150 mL water. The three jars containing the PSDs were closed and agitated for a period of 14 days on a shaker table. After 14 days, the PSDs at the marine site were collected by divers, brought to the surface, placed in a shipping container, and sent to a laboratory, where they were wiped with moistened tissues, placed in small vials, and extracted with 10 mL hexane solvent. The three PSDs deployed in the 8-oz jars (under agitated conditions) were also retrieved and processed in the same manner. The PSDs containing Sudan III that were not deployed (i.e., were not placed in the sediment, but retained in storage) were also extracted in the same manner. The solvents from the PSDs were analyzed on a benchtop UV/Vis spectrophotometer at wavelengths of 465 nm (to indicate the concentration of Sudan III in the solvent). Concentrations of these dyes in the PE could be calculated assuming a known mass of each PE piece.

Using the average concentrations of Sudan III in the unexposed PSDs (to represent the t=0 concentration) and the concentration of Sudan III in the PSDs that had been exposed to the marine sediment and agitated sediment for 14 days (the time t concentrations), one could estimate the kₑ values using the equation provided in Example 1. The average kₑ values were found to be 0.030 and 0.094 day⁻¹ for the marine sediment and agitated sediment, respectively. The approximate factor of 3 difference in these average kₑ values indicates that the agitation technique (shaker table) would increase the sampling rate for analytes with a similar hydrophobicity of Sudan III by approximate factor of 3. This increase in sampling rate afforded by agitation improves the detection limits and lowers the uncertainty of the passive sampling approach, as noted by Ghosh et al. (supra). Thus, the dye approach can be used to quantify the increase in sampling rates for various methods (i.e., unagitated deployments, various types of agitated deployments), and provides a low cost and efficient method compared to other PRCs.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A visibly detectable performance reference compound for use in a passive sampling device to detect an analyte of interest in an environment.

2. The compound of claim 1, wherein the compound is absorbable by a material selected from the group consisting of acetate, polyacrylate, silicone rubber, low-density polyethylene (LDPE), polyethylene (PE), polyoxymethylene (POM), polydimethylsiloxane (PDMS), polyethersulfone (PES), and/or solid phase microextraction fibers coated with such sorbents.

3. The compound of claim 1, wherein the compound is hydrophobic or hydrophilic.

4. The compound of claim 1, wherein the compound emits a wavelength from about 0.01 nanometers to about 1.0 micrometers.

5. The compound of claim 1, wherein the compound emits a wavelength from about 300 nanometers to about 700 nanometers,

6. The compound of claim 1, wherein the compound has a first octanol/water partition coefficient (K_{OW}) or a first log K_{OW} and the analyte has a second K_{OW} or a second log K_{OW}, wherein the first K_{OW} or the first log K_{OW} and the second K_{OW} or the second log K_{OW} are between about 0.01% to about 10% of each other.

7. The compound of claim 1, wherein the compound has a first octanol/water partition coefficient (K_{OW}) and the analyte has a second K_{OW}, wherein the first K_{OW} or the first log K_{OW} and the second K_{OW} or the second log K_{OW} are between about 0.01% to about 5% of each other.

8. The compound of claim 1, wherein the compound has a first PSD/water partition coefficient and the analyte has a second PSD/water partition coefficient, wherein the first PSD/water partition coefficient and the second PSD/water partition coefficient are between about 0.01% to about 10% of each other.

9. The compound of claim 1, wherein the compound has a first PSD/water partition coefficient and the analyte has a second PSD/water partition coefficient, wherein the first PSD/water partition coefficient and the second PSD/water partition coefficient are between about 0.01% to about 5% of each other.

10. The compound of claim 1, wherein the analyte of interest is an organic molecule.

11. The compound of claim 1, wherein the environment is selected from the group consisting of liquid, solid, and gas.

12. The compound of claim 1, wherein the compound is not radioactive.

13. A passive sampling device comprising the compound of claim 1.

14. A kit comprising a first visibly detectable performance reference compound and a second visibly detectable performance reference compound for use in a passive sampling device to detect an analyte of interest in an environment, the first compound having a first property selected from the group consisting of a first octanol/water partition coefficient (K_{OW}), a first log K_{OW}, and a first PSD/water partition coefficient, the second compound having a second property selected from the group consisting of a second K_{OW}, a second log K_{OW}, and a second PSD/water partition coefficient, and the analyte having a third property selected from the group consisting of a third K_{OW}, a third log K_{OW}, or a third PSD/water partition coefficient, wherein the third property is between the first property and the second property.

15. The kit of claim 14, wherein at least one of the first visibly detectable performance reference compound and the second visibly detectable performance reference compound is absorbable by a material selected from the group consisting of acetate, polyacrylate, silicone rubber, low-density polyethylene (LDPE), polyethylene (PE), polyoxymethylene (POM), polydimethylsiloxane (PDMS), polyethersulfone (PES), and/or solid phase microextraction fibers coated with such sorbents

16. The kit of claim 14, wherein at least one of the first visibly detectable performance reference compound and the second visibly detectable performance reference compound is hydrophobic or hydrophilic.

17. The kit of claim 14, wherein at least one of the first visibly detectable performance reference compound and the second visibly detectable performance reference compound emits a wavelength from about 0.001 nanometers to about 1.0 micrometers.

18. The kit of claim 14, wherein at least one of the first visibly detectable performance reference compound and the second visibly detectable performance reference compound emits a wavelength from about 300 nanometers to about 700 nanometers,

19. The kit of claim 14, wherein the analyte of interest is an organic molecule.

20. The kit of claim 14, wherein the environment is selected from the group consisting of liquid, solid, or gas.

21. The kit of claim 14, wherein at least one of the first visibly detectable performance reference compound and the second visibly detectable performance reference compound is not radioactive.

22. A kit comprising a first passive sampling device comprising the first visibly detectable performance reference compound of claim 14 and a second passive sampling device comprising the second visibly detectable performance reference compound of claim 14.

23. A method for detecting an analyte suspected to be in an environment, the analyte having an analyte octanol/water partition coefficient (K_{OW}) and an analyte PSD/water partition coefficient, comprising:
(a) contacting the environment for a time period with a passive sampling device comprising visibly detectable performance reference compound, the compound having a compound property selected from the group consisting of a compound octanol/water partition coefficient (K_{OW}), a compound log K_{OW}, and a compound PSD/water partition coefficient that is between about 0.01% to about 10% of a property of the analyte, the analyte property selected from the group consisting of an analyte K_{OW}, an analyte log K_{OW} or an analyte PSD/water partition coefficient; and
(b) after the time period, measuring an amount of compound in the passive sampling device, wherein the amount of compound in the passive sampling device is correlative with the amount of analyte in the environment.

24. The method of claim 23, wherein step (b) comprises extracting the compound from the passive sampling device into a solvent and measuring the amount of compound in the solvent.

25. The method of claim 23, wherein the compound property is between about 0.01% to about 5% of the analyte property.
